(19) United States
(12) Patent Application Publication
(10) Pub. No.: US 2024/0123142 A1
(43) Pub. Date: Apr. 18, 2024

(54) Device for Subcutaneous Delivery of a Medicament

(71) Applicant: Nuova OMPI S.r.l. Unipersonale, Padua (IT)

(72) Inventors: Mattia Cattaneo, Limbiate (MB) (IT); Pasquale Cirulli, Vermezzo con Zelo (MI) (IT); Paolo Degan, Ranica (BG) (IT); Andrea Giacomozzi, Milano (MI) (IT); Paolo Golfetto, Preganziol (TV) (IT); Tommaso Borghi, Milano (MI) (IT); Gustavo de Souza Messias, Milano (MI) (IT); Marco Pirinoli, Cusano Milanino (MI) (IT); Christian Riva, Cinisello Balsamo (MI) (IT); Nicola Bonserio, Giovinazzo (BA) (IT); Ilaria Nicoli, Parma (PR) (IT)

(21) Appl. No.: 18/486,867

(22) Filed: Oct. 13, 2023

(30) Foreign Application Priority Data
Oct. 14, 2022 (IT) 202022000004167
Oct. 14, 2022 (IT) 202022000004179
Oct. 14, 2022 (IT) 202022000004182

(51) Int. Cl. A61M 5/142; A61M 5/172

(52) U.S. Cl. CPC A61M 5/14244; A61M 5/172

(57) ABSTRACT

A device for subcutaneous delivery of a medicament includes a delivery module to house a cartridge containing a medicament to be delivered to a patient via a needle and a control module coupled to the delivery module to house a control unit and a battery to power the control unit. The delivery module includes an application surface that contacts the patient's body and two opposite first gripping perimeter surfaces extending along a first direction parallel to the application surface and along a second direction perpendicular to the application surface and having substantially equal dimensions in the second direction. The control module includes two opposite second gripping perimeter surfaces extending along the first direction and along the second direction and having substantially equal dimensions in the second direction. The two first gripping perimeter surfaces and the two second gripping perimeter surfaces have substantially equal dimensions in the first direction.

### CROSS REFERENCE TO RELATED APPLICATIONS

The present application claims priority to Italian Utility Model Application No. 202022000004167, filed on Oct. 14, 2022, Italian Utility Model Application No. 202022000004179, filed Oct. 14, 2022, and Italian Utility Model Application No. 202022000004182, filed Oct. 14, 2022, the disclosures of which are incorporated herein by reference in their entirety.

### TECHNICAL FIELD

The present disclosure relates to a device for subcutaneous delivery of a medicament. In particular, the present disclosure relates to a device configured to be applied on the patient's body in order to allow the subcutaneous delivery of a predetermined dose of medicament.

### BACKGROUND

Outpatient medicament delivery devices can include two mutually couplable modules, such as a module that includes, inter alia, a cartridge containing a medicament to be delivered and another module that includes, inter alia, a control unit of the device and the battery for the power supply of the control unit. In order to allow the delivery of the medicament and to avoid damaging one or both of the aforementioned modules, it is necessary that these modules are mutually coupled correctly. However, the small size of the delivery devices can make it difficult for the users to grip and couple the modules of the delivery devices, especially in the case of elderly people or people with reduced limb mobility (for example due to arthritis or Parkinson).

### SUMMARY OF THE DISCLOSURE

The present disclosure relates to devices for subcutaneous delivery of a medicament, including a delivery module and a control module reversibly couplable to the delivery module. In order to optimize the grip of the two modules, both modules of the present disclosure can have opposite gripping surfaces having equal surface extent.

The present disclosure therefore relates, in a first aspect thereof, to a device for subcutaneous delivery of a medicament, comprising:
a delivery module configured to house a cartridge containing a medicament to be delivered to a patient via a needle;
a control module reversibly couplable to the delivery module and configured to house at least one control unit and a battery to power the control unit;
   wherein the delivery module comprises:
an application surface configured to contact the patient's body;
two opposite first gripping perimeter surfaces extending along a first direction parallel to the application surface and along a second direction perpendicular to the application surface, wherein the two first gripping perimeter surfaces have substantially equal dimensions in the second direction;
   wherein the control module comprises:
two opposite second gripping perimeter surfaces extending along the first direction and along the second direction, wherein the two second gripping perimeter surfaces have substantially equal dimensions in the second direction;
   wherein the two first gripping perimeter surfaces and the two second gripping perimeter surfaces have substantially equal dimensions in the first direction.

This, and other aspects, can include one or more of the following features. The delivery module can include opposite coupling recesses extending along the first direction close to the first gripping perimeter surfaces, and the control module can include opposite coupling projections extending along the first direction close to the second gripping perimeter surfaces and configured to slide in the coupling recesses during the coupling of the control module to the delivery module. The two first gripping perimeter surfaces can be specular with respect to a first central plane of the delivery module extending along the first direction and the second direction, the two second gripping perimeter surfaces can be specular with respect to a second central plane of the control module extending along the first direction and the second direction, and the control module can be coupled to the delivery module, where the first central plane coincides with the second central plane. The delivery module can include a third perimeter surface extending between the two first gripping perimeter surfaces along the second direction and along a third direction that is parallel to the application surface and perpendicular to the first direction, the control module can include a fourth perimeter surface extending between the two second gripping perimeter surfaces along the second direction and the third direction, the fourth perimeter surface being parallel to the third perimeter surface when the control module is coupled to the delivery module, and the sum of the dimensions of any one of the first gripping perimeter surfaces and of any one of the second gripping perimeter surfaces along the first direction is smaller than the dimension of the third perimeter surface and of the fourth perimeter surface along the third direction. The delivery module can include a first interface surface and the control module comprises a second interface surface substantially counter-shaped to the first interface surface and configured to be arranged so as to face the first interface surface when the control module is coupled to the delivery module, the first interface surface can include a first interface portion extending from one of the first gripping perimeter surfaces to the other first gripping perimeter surface and having a concave profile, and the second interface surface can include a second interface portion having a convex profile and configured to be arranged so as to face the first interface portion when the control module is coupled to the delivery module. The concave profile can be defined by two opposite surfaces parallel to the two first gripping perimeter surfaces and a connection surface parallel to the third perimeter surface, and the convex profile can be defined by two opposite surfaces parallel to the two second gripping perimeter surfaces and a connection surface parallel to the fourth perimeter surface. Each of the two opposite surfaces of the concave profile can be closer to a respective first gripping perimeter surface than to the first central plane of the delivery module, and each of the two opposite surfaces of the convex profile can be closer to the first gripping perimeter surface than to the second central plane of the control module when the control module is coupled to the delivery module. The connection surface of the concave profile and the connection surface of the convex profile can be closer to the third perimeter surface than a center plane of the device extending along the second direction and the third direction when the control module is coupled to the delivery module. The first interface portion can be defined in a top portion of the delivery module and the second interface portion can be defined in a top portion of the control module. The first interface surface can include a first circular portion and the second interface surface can include a second circular portion configured to overlap the first circular portion when the control module is coupled to the delivery module. The first circular portion can define a recess in the delivery module and the second circular portion can define a protrusion in the control module. The first interface surface can include a second interface portion substantially parallel to the application surface and a third interface portion extending from the second interface portion away from the application surface up to the first interface portion. The first interface portion, the second interface portion and the third interface portion of the first interface surface can have substantially equal dimensions in the third direction. The device can include at least one gasket interposed between at least part of the first interface surface and at least part of the second interface surface. The control module can have a colour different from that of the delivery module. The control module can have a surface finish different from that of the delivery module.

Such a device allows a stable and secure grip of both modules by the user, even in the case of particularly miniaturized modules.

It is desirable to prevent an unintentional or intentional decoupling of the two modules after the device has been applied on the patient's body for the delivery of the medicinal product and during said delivery. A decoupling of such modules at this time may result in the non automatic retraction of the cutaneous needle from the patient's body. In this case, a manual extraction of the cutaneous needle should be carried out, with inevitable discomfort for the patient. The automatic retraction phase of the cutaneous needle is optimized to limit the pain perceived by the patient during the exit of the needle from the patient's body.

The device of the present disclosure is suitable for preventing an unintentional or intentional decoupling of the two modules after the device has been applied on the patient's body for the delivery of the medicament and during said delivery.

An example device comprising a solution suitable for preventing the unintentional/intentional decoupling of the two modules is described in US 2022/0031940A1.

The present disclosure therefore relates, in a further aspect thereof, to a device for subcutaneous delivery of a medicament, comprising:
a delivery module configured to house a cartridge containing a medicament to be delivered to a patient via a needle;
a control module reversibly couplable to the delivery module and configured to house at least one control unit and a battery to power the control unit;
   wherein one of the delivery module or the control module comprises a locking projection, and
   wherein the other of the delivery module or the control module comprises:
a seat having an inlet configured to receive the locking projection during the coupling between the delivery module and the control module and an internal volume configured to house the locking projection at the end of the coupling between the delivery module and the control module;
a slider associated with the seat and slidably movable between a first position in which a service portion of the slider obstructs the inlet, hindering the passage of the locking projection from the inlet to the internal volume, and a second position in which the service portion does not obstruct the inlet, allowing the passage of the locking projection from the inlet to the internal volume;
   wherein the control module is stably coupled to the delivery module when the locking projection is housed in the internal volume and the slider is in the first position.

This, and other aspects, can include one or more of the following features. The control module can include the locking projection and the delivery module can include the seat and the slider. The device can include an elastic element interposed between the slider and the seat and configured to retain the slider in the first position both before the coupling between the delivery module and the control module and at the end of the coupling between the delivery module and the control module. The elastic element can be interposed between a first shoulder formed in the seat and a second shoulder formed in the slider. The slider can include a main body slidably mounted in the seat and a manoeuvring portion which is manually operable for shifting the main body from the first position to the second position. The manoeuvring portion can project with respect to the seat. The delivery module can include an application surface configured to be faced towards the patient's body when the device is applied on the patient, wherein the manoeuvring portion is arranged at the application surface. The manoeuvring portion can be movable along a manoeuvring direction and wherein the control module can be slidably couplable to the delivery module along a coupling direction perpendicular to the manoeuvring direction. The manoeuvring direction and the coupling direction can be parallel to the application surface. The seat can include a coupling portion defined between two opposite guide surfaces that extend from the inlet to the internal volume along the coupling direction and are configured to guide the locking projection between the inlet and the internal volume. When the slider is in the first position, the service portion can be arranged at the coupling portion, and when the slider is in the second position, the service portion can be arranged in a position adjacent to the coupling portion. The coupling portion can include a stop surface extending perpendicularly to the coupling direction and facing the internal volume. The service portion can include a push surface facing the inlet when the slider is in the first position and oriented obliquely with respect to the coupling direction. The service portion can include a locking surface facing the internal volume when the slider is in the first position and oriented perpendicularly with respect to the coupling direction. The locking surface can be parallel to the stop surface. When the control module is stably coupled to the delivery module, the locking projection can be surrounded by the stop surface, the opposite guide surfaces, and the locking surface. The delivery module can include a first interface surface and the control module can include a second interface surface configured to be arranged so as to face the first interface surface when the control module is coupled to the delivery module, and the locking projection can extend from the second interface surface along a development direction perpendicular to the coupling direction. The locking projection can include a push portion extending along the development direction and a hooking portion extending from the push portion along a direction parallel to the coupling direction, where the seat can include a hooking surface configured to be arranged in abutment against the hooking portion when the locking projection is housed in the internal volume. The control module can include a closing projection extending from an edge of the second interface surface and configured to close the inlet when the control module is coupled to the delivery module. The closing projection can extend along a direction parallel to the push portion.

In the present description and in the subsequent claims, the term “stably coupled” is used to indicate a coupling between the delivery module and the control module such that in order to be able to decouple the two modules, both a relative movement of the two modules and a further action required to allow such relative movement are necessary.

In the aforementioned device, the coupling between the two modules initially includes the mutual movement of the two modules to be arranged close to each other and the insertion of the locking projection provided in one of the two modules into the seat provided in the other of the two modules. This coupling ends when the locking projection is positioned inside the internal volume of the seat. At this time, positioning the slider in its first position guarantees the stability of this coupling.

The decoupling of the two modules comprises at first shifting the slider to its second position and then moving the two modules away from each other. As two distinct actions are required, the unintentional decoupling of the two modules is prevented and the voluntary decoupling of the two modules is made more complicated.

Some outpatient type subcutaneous delivery devices have an inspection window to allow the inspection of the medicament that is inside the cartridge before starting the delivery of the medicament. In fact, it is advisable the medicament to be delivered to the patient only after having verified, by visual check, that the medicament is unaltered. Examples of outpatient type subcutaneous delivery devices with an inspection window are described in WO 2006/024650A2, WO 2003/099358A2, WO 2006/077262A1. In all of the aforementioned examples, the inspection window is positioned on an external surface of the delivery device. In particular, in the devices described in WO 2006/024650A2 and WO 2003/099358A2 the inspection window is arranged on the external surface facing away from the patient's body after the delivery device has been applied on the patient's body, whereas in WO 2006/077262A1 the inspection window is arranged on the external surface facing towards the patient's body after the delivery device has been applied on the patient's body. In devices in which the inspection window is arranged on the external surface intended to be faced away from the patient's body, such as for example in the devices described in WO 2006/024650A2 and WO 2003/099358A2, the inspection window is always accessible to the user's view (in the following this condition will also be briefly indicated with the term “visible”). This causes a number of drawbacks, some of which are discussed below.

A first drawback is correlated to the fact that, typically, a control interface of the delivery device, such as for example one or more buttons or one or more LEDs, is also provided on said external surface. In this case, during the application of the delivery device on the patient's body, the user (especially a non-expert user) could not immediately identify the correct orientation to be given to the delivery device in order to position it correctly on the patient's body. Indeed, the user may wonder, for example, whether to orient the delivery device in order to facilitate visual access to the inspection window or to the control interface, thus risking to opt for a position other than the correct one. In this case, the user might not be able to see possible error messages that are communicated through the control interface.

Another drawback is correlated to the fact that, since the inspection window is visible, the patient would be able to inspect the medicament that is within the cartridge even at the end of the delivery cycle. Consequently, if, for example, the delivery therapy did not provide for the delivery of the entire dose of medicament that is within in the cartridge, the patient, by seeing that not all the medicament that is within the cartridge has been delivered, could worry, without there actually being anything abnormal.

In those devices in which the inspection window is arranged on the external surface intended to be faced towards the patient's body, such as for example in the device described in WO 2006/077262A1, during the delivery of the medicament the patient could decide to move the delivery device to have a visual access to the inspection window and verify, perhaps only out of curiosity, the amount of medicament which has been delivered and/or the amount that is still present in the cartridge. However, it would be advisable instead to avoid any movement of the delivery device once it has been applied on the patient's body, so as not to risk incurring various problems, such as for example the accidental detachment of the device from the patient's body or the breakage of the needle inserted into the patient's body.

Therefore, there exists a need to provide a subcutaneous delivery device in which the inspection window is arranged in such a position as to be visible only before the start of the delivery therapy and not also during delivery or at the end of the delivery, when the device is still applied on the patient's body. In order to meet this need it is appropriate for the delivery device to be made up of two distinct modules, one configured to house the cartridge and the other configured to house command components of the device, in particular a control unit and the battery for the power supply of said control unit, said modules being further configured to be mutually coupled at respective interface surfaces which, after having coupled the two modules, have respective surface portions that are not visible.

Therefore, the inspection window of the present disclosure is arranged on the interface surface of the module in which the cartridge is housed and at a portion of the surface that is covered by the other interface surface when the two modules are mutually coupled. In this way, the inspection window is visible only before the two modules are mutually coupled, while it is not accessible, not even visually, during the delivery of the medicament and at the end of the delivery when the two modules are still coupled to each other.

The present disclosure therefore relates, in a further aspect thereof, to a device for subcutaneous delivery of a medicament, comprising:
a delivery module configured to house a cartridge containing a medicament to be delivered to a patient via a needle;
a control module reversibly coupled to the delivery module and configured to house at least a control unit and a battery to power the control unit;
   wherein the delivery module comprises a first interface surface facing the control module and the control module comprises a second interface surface facing the first interface surface;
   wherein the first interface surface comprises an inspection window covered by the second interface surface and configured to allow a visual inspection of the cartridge when the control module is decoupled from the delivery module.

This, and other aspects, can include one or more of the following features. The delivery module can include an application surface configured to come into contact with the patient's body, and the delivery module can house the cartridge between the application surface and the inspection window. The inspection window can be parallel to the application surface. The application surface can include a light-permeable portion configured to allow an external light to reach the cartridge when the application surface is not in contact with the patient's body. The light-permeable portion and the inspection window can be mutually overlapped. The light-permeable portion can be greater than the inspection window. The application surface can include an opening configured to allow the insertion of the cartridge into the delivery module during the assembly of the delivery module, and the light-permeable portion can include a transparent element fixed at the opening to occlude the opening. The application surface can include an adhesive layer configured to attach the delivery module to the patient's skin, a portion of the adhesive layer being placed on the transparent element, the adhesive layer being configured to allow an external light to reach the cartridge when the application surface is not in contact with the patient's body. The adhesive layer can be non-transparent.

Such a device prevents the user from any possibility of access to the inspection window after the delivery device is coupled to the patient's body. The inspection window is in fact only accessible before coupling the delivery module and the control module together. To have access to the inspection window after having coupled the two modules, it is necessary to decouple them, an operation that is done only at the end of the delivery of the therapy.

The device of the disclosure can comprise one or more of the following features, taken individually or possibly combined with each other.

In some embodiments, the delivery module is disposable.

In some embodiments, the control module is reusable.

In some embodiments, the delivery module comprises opposite coupling recesses extending along the first direction close to said first gripping perimeter surfaces and said control module comprises opposite coupling projections extending along the first direction close to said second gripping perimeter surfaces and configured to slide in said coupling recesses during the coupling of the control module to the delivery module. The coupling recesses therefore act as guides for the coupling projections, making it extremely easy to achieve a particularly precise, as well as correct, mutual coupling of the two modules.

In some embodiments, said two first gripping perimeter surfaces are specular with respect to a first central plane of said delivery module extending along the first direction and the second direction. In this way, gripping the delivery module is particularly easy.

In some embodiments, said two second gripping perimeter surfaces are specular with respect to a second central plane of said control module extended along the first direction and the second direction. In this way, gripping the control module is particularly easy.

In some embodiments, when said control module is coupled to said delivery module said first central plane coincides with said second central plane.

In some embodiments, said delivery module comprises a third perimeter surface extending between said two first gripping perimeter surfaces along the second direction and along a third direction parallel to the application surface and perpendicular to the first direction.

In some embodiments, said control module comprises a fourth perimeter surface extending between said two second gripping perimeter surfaces along the second direction and the third direction.

In some embodiments, said fourth perimeter surface is parallel to the third perimeter surface when the control module is coupled to the delivery module.

In some embodiments of the device of the present disclosure, the sum of the dimensions of any one of said first gripping perimeter surfaces and of any one of said second gripping perimeter surfaces along said first direction is smaller than the dimension of said third perimeter surface and of said fourth perimeter surface along the third direction.

In this case, the device has a main dimension along the third direction, that is along a direction substantially orthogonal to that of the opposite gripping surfaces, allowing the users with reduced limb mobility not to have to bring their thumb and index finger to each other to safely and stably handle the device.

In some embodiments, the third perimeter surface and the fourth perimeter surface have substantially equal dimensions in the third direction.

In some embodiments, the delivery module comprises a first interface surface and the control module comprises a second interface surface that is substantially counter-shaped to the first interface surface and configured to be arranged so as to face the first interface surface when the control module is coupled to the delivery module. The provision on the two modules of mutually counter-shaped interface surfaces suggests to the user the correct relative orientation to be given to the two modules before proceeding with their mutual coupling.

In some embodiments, the first interface surface comprises a first interface portion extending from one of the first gripping perimeter surfaces to the other first gripping perimeter surface.

In some embodiments, said first interface portion has a concave profile.

In some embodiments, the second interface surface comprises a second interface portion configured to be arranged so as to face said first interface portion when the control module is coupled to the delivery module.

In some embodiments, said second interface portion has a convex profile.

The concave and convex shape of the portions of the interface surfaces of the delivery module and of the control module, respectively, are particularly suitable to suggest to the user the correct orientation of the two modules before their mutual coupling and to guide the user during the mutual coupling of the two modules.

In some embodiments, the concave profile is defined by two opposite surfaces parallel to the two first gripping perimeter surfaces and a connection surface parallel to the third perimeter surface.

In some embodiments, the convex profile is defined by two opposite surfaces parallel to the two second gripping perimeter surfaces and a connection surface parallel to the fourth perimeter surface.

In some embodiments of the device of the present disclosure, each of the two opposite surfaces of the concave profile is closer to a respective first gripping perimeter surface than to the first central plane of said delivery module.

In some embodiments, each of the two opposite surfaces of the convex profile is closer to said first gripping perimeter surface than to the second central plane of said control module when the control module is coupled to the delivery module.

In this way, the concave and convex profiles have a sufficiently wide surface extension, to the benefit of the user who is thus facilitated in achieving the correct mutual orientation of the two modules before their coupling.

In some embodiments, the connection surface of the concave profile and the connection surface of the convex profile are closer to the third perimeter surface than to a center plane of the device extending along the second direction and the third direction when the control module is coupled to the delivery module.

In other words, the connection surface of the concave profile is closer to the third perimeter surface than to the aforementioned center plane and the connection surface of the convex profile is farther from the fourth perimeter surface than the aforementioned center plane when the control module is coupled to the delivery module.

In some embodiments, said first interface portion is defined in a top portion of the delivery module and said second interface portion is defined in a top portion of the control module.

In the present description and in the subsequent claims, the term “top portion” is used to indicate a portion of the delivery module which, when the delivery module is applied to the patient's body, is farther from the application surface than the cartridge and a portion of the control module which, when the control module is coupled to the delivery module and the device thus assembled is applied on the patient's body, is farther from the application surface than the control unit and/or the battery for the power supply of the control unit.

In some embodiments, the first interface surface comprises a first circular portion and the second interface surface comprises a second circular portion configured to overlap the first circular portion when the control module is coupled to the delivery module. These circular portions are configured to allow a driving rotor provided in the control module at the second circular portion to rotatably actuate by magnetic coupling a magnetic rotor provided in the delivery module at the first circular portion.

In some embodiments, said first circular portion defines a recess in the delivery module and said second circular portion defines a protrusion in the control module. In this way, a precise alignment between the aforementioned rotors is achieved.

In some embodiments, the first interface surface comprises a second interface portion substantially parallel to the application surface.

In the present description and in the subsequent claims, a surface is meant to be “substantially parallel” to another both when each of the two surfaces extends on a plane parallel to the plane in which the other surface extends and when at least one of the two surfaces comprises portions of planar surfaces arranged on different planes and parallel to the other surface or to portions of planar surfaces of the other surface.

In some embodiments, the first interface surface comprises a third interface portion extending from the second interface portion of the first interface surface away from to the application surface up to the first interface portion of the first interface surface.

In some embodiments, the first interface portion and/or the second interface portion and/or the third interface portion of the first interface surface have substantially equal dimensions in the third direction.

In some embodiments, at least one gasket is interposed between at least part of said first interface surface and at least part of said second interface surface. This is in order to ensure the tightness of the coupling between the two modules.

In some embodiments, said control module has a colour different from that of said delivery module, so as to allow the user an immediate recognition of each of the two modules.

In some embodiments, said control module has a surface finish different from that of said delivery module. This arrangement also facilitates the recognition of each of the two modules by the user.

In some embodiments, the control module comprises the locking projection and the delivery module comprises said seat and said slider.

In some embodiments, the aforementioned device comprises an elastic element interposed between the slider and said seat and configured to retain said slider in said first position both before the coupling between the delivery module and the control module and at the end of the coupling between the delivery module and the control module. This elastic element allows shifting the slider from its first position to its second position during the coupling of the two modules and the automatic return of the slider to its second position at the end of the coupling of the two modules, i.e. when the locking projection is arranged in the internal volume of the seat.

In some embodiments, said elastic element is interposed between a first shoulder formed in the seat and a second shoulder formed in the slider.

In some embodiments, the slider comprises a main body slidably mounted in said seat and a manoeuvring portion which is manually operable for shifting the main body from the first position to the second position.

In some embodiments, said manoeuvring portion projects with respect to said seat. This expedient facilitates reaching the manoeuvring portion of the slider when the user wants to decouple the two modules.

In some embodiments, the delivery module comprises an application surface configured to be faced towards the patient's body when the application device is applied on the patient.

In some embodiments, said manoeuvring portion is arranged at said application surface, i.e. in a position that is physically not accessible to the user when the device is applied on the patient's body for the delivery of the therapy. This avoids the risk of allowing unintentional/intentional decoupling of the two modules.

In some embodiments, said manoeuvring portion is movable along a manoeuvring direction and the control module is slidably couplable to the delivery module along a coupling direction perpendicular to the manoeuvring direction. In this way, the decoupling of the two modules cannot take place simply after the modules are moved away from each other.

In some embodiments, said manoeuvring direction and said coupling direction are parallel to the application surface.

In some embodiments, said seat comprises a coupling portion defined between two opposite guide surfaces that extend from said inlet to the internal volume along the coupling direction and are configured to guide the locking projection between the inlet and the internal volume.

In some embodiments, when the slider is in the first position the service portion is arranged at said coupling portion and when the slider is in the second position the service portion is arranged in a position adjacent to said coupling portion. Thus, the service portion obstructs the passage of the locking projection through the seat coupling portion when the slider is in its first position and allows the passage of the locking projection through the seat coupling portion when the slider is in its first position.

In some embodiments, said coupling portion comprises a stop surface extending perpendicularly to the coupling direction and facing the internal volume. This stop surface defines an end stop for the locking projection when it enters the internal volume after having passed through the coupling portion.

In some embodiments, said service portion comprises a push surface facing said inlet when the slider is in the first position and oriented obliquely with respect to said coupling direction. The movement of the slider can therefore take place following the push exerted by the locking projection on the push surface of the slider during the movement of the two modules to be arranged close to each other. The oblique arrangement of the push surface with respect to the coupling direction allows the locking projection to exert a push on the slider in a direction orthogonal to the coupling direction, so as to shift the slider between its first position and its second position and allow the coupling of the two modules.

In some embodiments, said service portion comprises a locking surface facing the internal volume when the slider is in the first position and oriented perpendicularly with respect to said coupling direction. Such a locking surface obstructs the access of the locking projection to the coupling portion, and hence to the inlet, when the slider is in its first position, ensuring the stability of the coupling.

In some embodiments, said locking surface is parallel to said stop surface.

In some embodiments, when the control module is stably coupled to the delivery module the locking projection is surrounded by said stop surface, said opposite guide surfaces and said locking surface. Thus, when the two modules are coupled the locking projection is enclosed between four distinct surfaces. This expedient makes the coupling of the two modules even more robust against accidental decoupling.

In some embodiments, the delivery module comprises a first interface surface and the control module comprises a second interface surface configured to be arranged so as to face the first interface surface when the control module is coupled to the delivery module.

In some embodiments, the locking projection extends from the second interface surface along a development direction perpendicular to the coupling direction.

In some embodiments, the locking projection comprises a push portion extending along said development direction and a hooking portion extending from the push portion along a direction parallel to the coupling direction.

In some embodiments, the seat comprises a hooking surface configured to be arranged in abutment against the hooking portion when the locking projection is housed in the internal volume.

In some embodiments, said control module comprises a closing projection extending from an edge of the second interface surface and configured to close said inlet when the control module is coupled to the delivery module, so as to prevent access to the slider from the inlet of the seat.

In some embodiments, said closing projection extends along a direction parallel to said push portion.

In some embodiments, the delivery module comprises an application surface configured not to come in contact with the patient's body. The delivery module therefore comprises two opposed external surfaces: one of these external surfaces defines the interface surface of the delivery module and is intended to face away from the body portion of the patient on which the delivery device is to be applied and to be covered by the interface surface of the control module when the latter is coupled to the delivery module, while the other of the aforementioned external surfaces defines the application surface of the delivery module and is intended to face the body portion of the patient on which the delivery device is to be applied.

In some embodiments, the delivery module is configured to house said cartridge between the application surface and the inspection window and, therefore, between the application surface and the interface surface of the delivery module.

In some embodiments, the inspection window is parallel to the application surface. This expedient allows an easy inspection of the medicament contained in the cartridge when the two modules are decoupled.

In some embodiments of the device of the present disclosure, the application surface comprises a light-permeable portion configured to allow an external light to reach the cartridge when the application surface is not in contact with the patient's body.

In some embodiments, the light-permeable portion and the inspection window are mutually overlapped. In this way, light entering the delivery module through the light-permeable portion of the application surface can reach the inspection window. The presence of the cartridge between the application surface and the inspection window does not hinder the aforementioned light to reach the inspection window since the cartridges are typically made of transparent material (and therefore of a material permeable to light) to allow the medicament contained therein to be seen.

In some embodiments, the light-permeable portion is greater than the inspection window. This expedient allows the inspection window to be reached by a relatively high amount of light.

In some embodiments, the application surface comprises an opening configured to allow the insertion of the cartridge into the delivery module during the assembly of the delivery module.

In some embodiments, the light-permeable portion comprises a transparent element fixed at said opening to occlude said opening. The aforementioned transparent element therefore acts as a closing door for the cartridge housing seat. This door, being transparent, allows the light to enter the delivery module to reach the cartridge and the inspection window.

In some embodiments, said transparent element is fixed at said opening by laser welding. This type of fixing is possible precisely because the closing door of the cartridge housing seat is transparent.

In some embodiments, the application surface comprises an adhesive layer configured to attach the delivery module to the skin of the patient.

In some embodiments, a portion of said adhesive layer is placed on said transparent element.

In some embodiments, said adhesive layer is configured to allow an external light to reach the cartridge when the application surface is not in contact with the patient's body. Thus, the aforementioned adhesive layer does not hinder the entry of light into the delivery module through the light-permeable portion of the application surface.

In some embodiments, said adhesive layer is not transparent, so as to prevent the user from being able to see the cartridge through the transparent element.

### BRIEF DESCRIPTION OF THE DRAWINGS

The features of the present disclosure will result from the following detailed description of some exemplary embodiments thereof, provided by way of non-limiting examples. Such a description will be made referring to the appended drawings, in which:

FIG. 1 shows a perspective view of a device for subcutaneous delivery of a medicament according to the present disclosure;

FIG. 2 shows a perspective view of a part of the device of FIG. 1;

FIG. 3 shows a bottom view of the part of FIG. 2;

FIG. 4 shows a sectional view of the part of FIG. 2;

FIG. 5 shows a perspective view of another part of the device of FIG. 1,

FIG. 6 shows a bottom view of the part of FIG. 5;

FIG. 7 shows a side view of the part of FIG. 5;

FIG. 8 shows an enlargement of some components of the device of FIG. 1 in a first operating configuration, with some parts hidden in order to show other parts;

FIG. 9 shows an enlargement of some components of the device of FIG. 1 in a second operating configuration, with some parts hidden in order to highlight other parts;

FIG. 10 shows an enlargement of some components of the device of FIG. 1 in a third operating configuration, with some parts hidden in order to highlight other parts.

### DETAILED DESCRIPTION

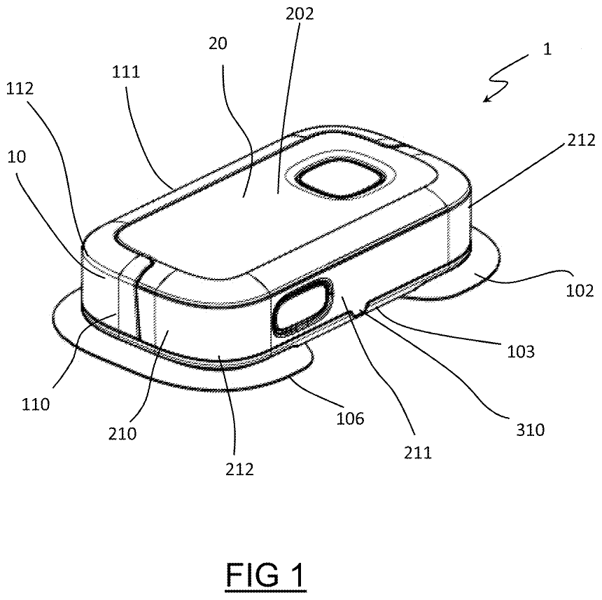

A device for subcutaneous delivery of a medicament is schematically shown in FIG. 1 and is indicated with reference numeral 1. The application of the device on the patient's body is carried out by a user, for example a doctor or a nurse or the patient.

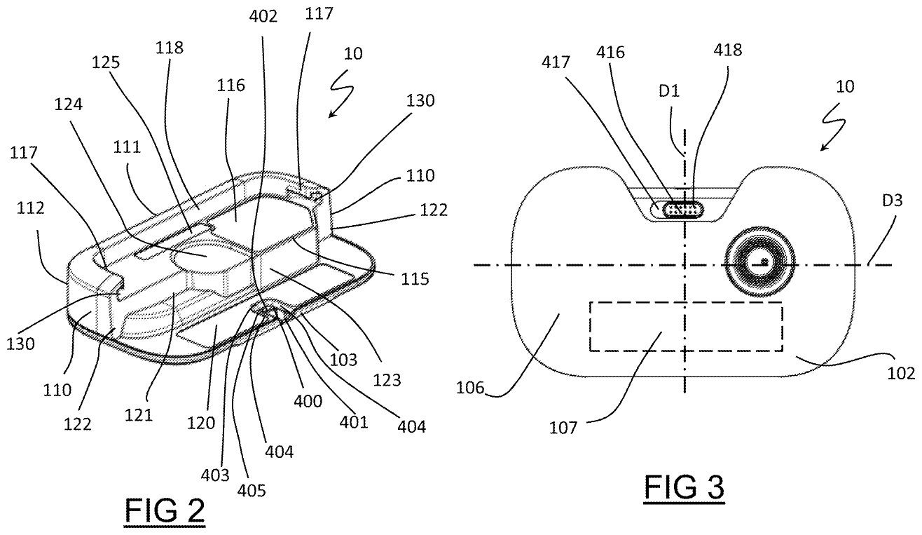

The device 1 comprises a delivery module 10, shown in FIGS. 2 to 4, and a control module 20, shown in FIGS. 5 to 7.

The device 1 is obtained by mutually coupling the delivery module 10 and the control module 20. As will become clear in the following of this description, this coupling is reversible, i.e. the two modules 10 and 20 after having been coupled can be decoupled.

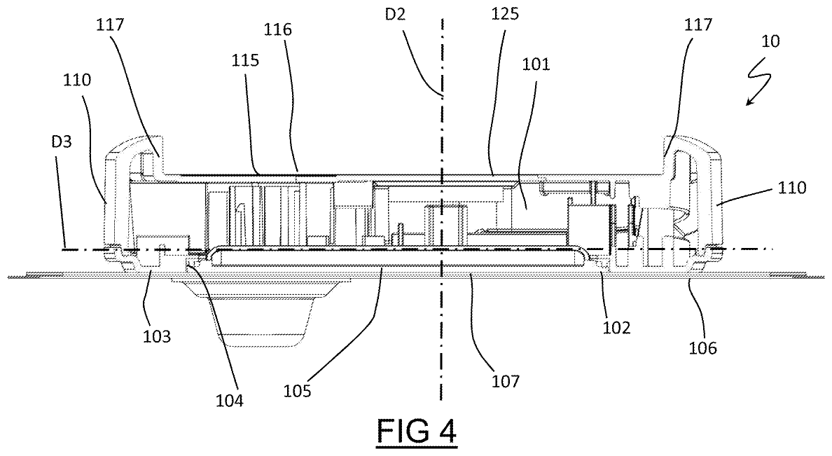

The delivery module 10 comprises an internal housing 101 (shown in FIG. 4) configured to house a cartridge. The cartridge comprises a cylindrical container made of a plastic or glass material containing a medicament to be delivered to a patient and a pierceable septum that closes the container and guarantees its sterility until the device 1 is used.

Prior to the use of the device 1, a fluidic path configured to allow passage of the medicament from the cartridge to the patient's body is associated with the delivery module 10. This fluidic path comprises a needle configured to pierce the septum (hereinafter referred to as the “septum needle”), a cutaneous needle configured to be inserted into the patient's body and a flexible tube that puts the aforementioned needles in fluid communication. In the present description, the term “fluidic path” is used to indicate any element or assembly that is configured to be connected, at a first end thereof, to the cartridge housed inside a device for subcutaneous delivery of a medicament and comprising, at an end thereof opposite to the aforementioned first end, a needle intended to be inserted into the patient's body, to allow the passage of the medicament from the cartridge to the patient's body via said needle.

The delivery module 10 further comprises a fluidic path opening system which is configured to move the septum needle and/or the cartridge so that the septum needle pierces the septum and enters in fluid communication with the interior of the cartridge so that medicament can be withdrawn therefrom.

The delivery module 10 also comprises a cutaneous needle insertion and retraction mechanism. This mechanism is configured to move the cutaneous needle before the start of the therapy by inserting it into the patient's body and at the end of the therapy by retracting it from the patient's body.

The delivery module 10 further comprises a drive chain configured to perform drug delivery following the opening of the fluidic path and the insertion of the cutaneous needle into the patient's body.

The delivery module 10 is, in some instances, disposable. In other words, the delivery module 10 is configured to be applied on the patient's body only once and for a certain period of time and to deliver, completely or in part, the content of the cartridge in one or more subsequent injections, even temporally spaced apart from each other. In some embodiments, the delivery module 10 does not provide for the possibility of refilling or replacing the cartridge and is configured to be disposed after removal from the patient's body.

The delivery module 10 comprises an application surface 102 configured to enter into contact with the patient's body. The application surface 102 is substantially flat.

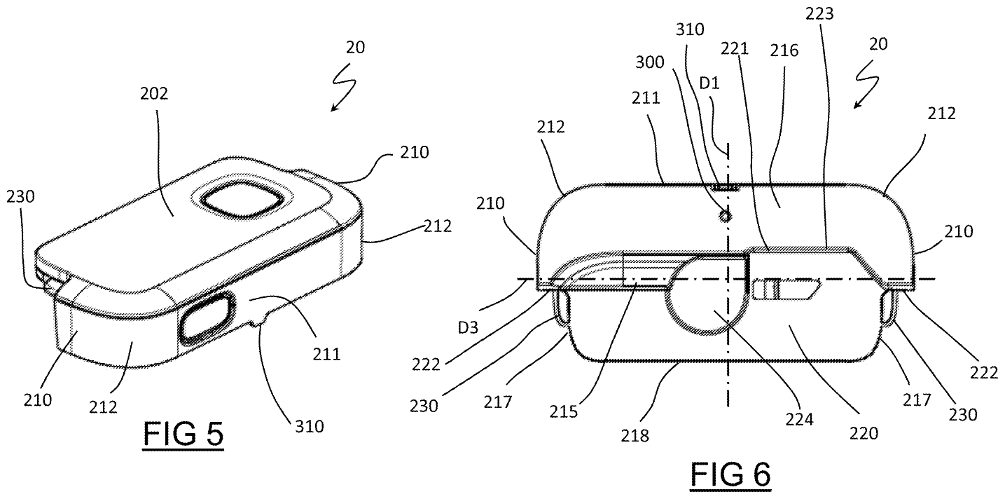

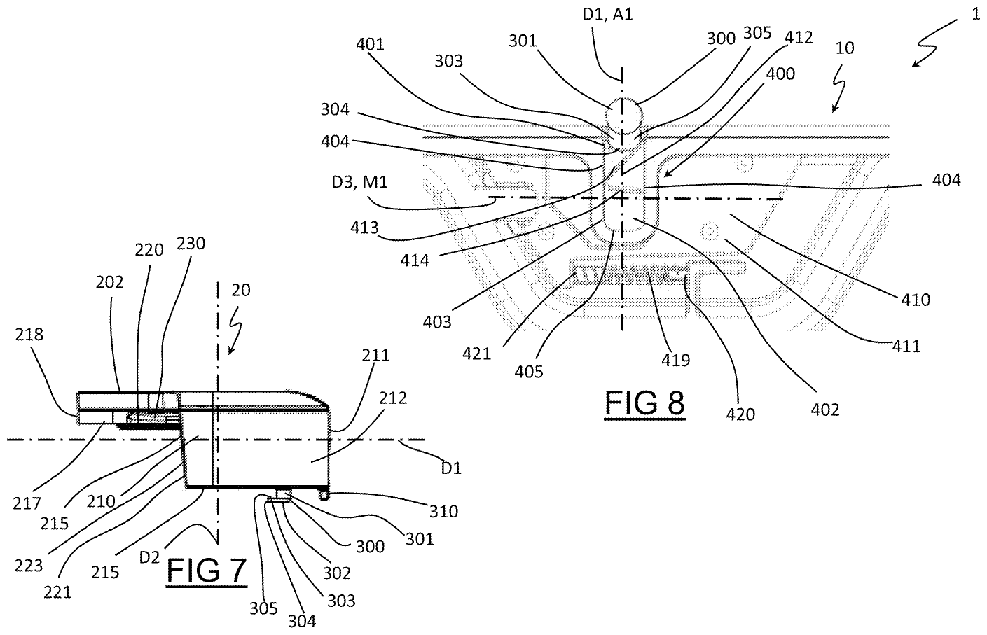

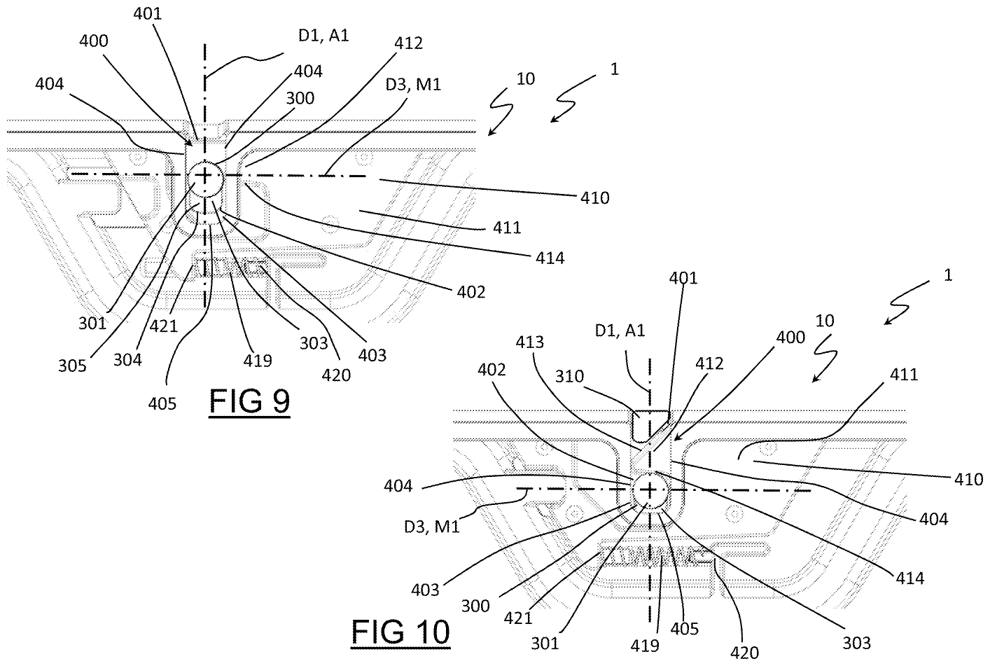

The application surface 102 defines a reference plane. With respect to the application surface 102, the following directions are defined: a first direction D1 (FIG. 3) parallel to the application surface 102 and to a coupling direction A1 of the two modules 10 and 20, a second direction D2 (FIG. 4) perpendicular to the application surface 102 and a third direction D3 (FIG. 3) parallel to the application surface 102 and perpendicular to the first direction D1 and to the second direction D2. When used with reference to the control module 20 or parts thereof, the first direction D1 (FIGS. 6, 8-10), the second direction D2 (FIG. 7) and the third direction D3 (FIGS. 6, 8-10) are to be understood as referred to a configuration of the control module 20 in which the control module 20 is coupled to the delivery module 10.

The application surface 102 comprises a base wall 103 having a substantially rectangular shape, with rounded corners. The base wall 103 has two short sides oriented in the first direction D1 and two long sides oriented in the third direction D3.

The base wall 103 is made of a rigid material, for example, a plastic material. The base wall 103 partially delimits the housing compartment 101 of the delivery module 10.

As shown in FIG. 4, the base wall 103 comprises an opening 104 configured to allow the cartridge to be positioned in the housing compartment 101 during the assembly of the delivery module 10.

The base wall 103 further comprises a transparent element 105, made for example of a transparent plastic material, fixed at the opening 104 in order to occlude it. The transparent element 105 is applied during the assembly phase after having introduced the cartridge into the housing compartment 101 through the opening 104 and is fixed at the opening 104 by laser welding.

The application surface 102 further comprises an adhesive layer 106 configured to attach the delivery module 10 to the patient's body. The adhesive layer 106 has an adhesive face attached to the base wall 103 and an opposite adhesive face configured to be attached to the patient's skin.

The adhesive layer 106 is configured to be crossed by light. The adhesive layer 106 covers the transparent element 105 and forms together with the latter a light-permeable portion 107 in the application surface 102. The light-permeable portion 107 is configured to allow the external light to reach the cartridge in the housing compartment 101 when the application surface is not in contact with the patient's body.

The adhesive layer 106 is not transparent and is configured to prevent seeing the cartridge in the housing compartment 101 through the light-permeable portion 107.

With reference to FIGS. 1 and 2, the delivery module 10 comprises two opposite first gripping perimeter surfaces 110. The first gripping perimeter surfaces 110 extend along the first direction D1 and along the second direction D2.

The first gripping perimeter surfaces 110 are adjacent to the application surface 102 and partially delimit the housing compartment 101 on opposite sides.

The first gripping perimeter surfaces 110 are placed at opposite short sides of the base wall 103 and form with the base wall 103 an angle substantially equal to 90°.

The first gripping perimeter surfaces 110 extend along the first direction D1 on a same long side of the base wall 103 towards the opposite long side moving on the respective short side.

The first gripping perimeter surfaces 110 extend in the first direction D1 over about half the extent of the short sides of the base wall 103.

The first gripping perimeter surfaces 110 extend from the base wall 103 along the second direction D2 away from the base wall 103.

The first gripping perimeter surfaces 110 have dimensions substantially equal to one another along the first direction D1 and the second direction D2. In particular, the two first gripping perimeter surfaces 110 are specular with respect to a first central plane of the delivery module 10 extending along the first direction D1 and the second direction D2.

Still with reference to FIGS. 1 and 2, the delivery module 10 comprises a third perimeter surface 111 extending along the second direction D2 and the third direction D3.

The third perimeter surface 111 is adjacent to the application surface 102 and partially delimits the housing compartment 101.

The third perimeter surface 111 is placed at one of the long sides of the base wall 103 and forms with the base wall 103 an angle substantially equal to 90°.

The third perimeter surface 111 extends from the base wall 103 along the second direction D2 away from the base wall 103.

The third perimeter surface 111 extends along the third direction D3 from a first gripping perimeter surface 110 to the other first gripping perimeter surface 110 moving on the respective long side of the base wall 103.

The third perimeter surface 111 has a dimension substantially equal to the dimension of the first gripping perimeter surfaces 110 in the second direction D2.

Each first gripping perimeter surface 110 is joined to the third perimeter surface 111 by a rounded joining portion.

As shown in FIGS. 2 and 4, the delivery module 10 comprises a first interface surface 115 configured to be arranged so as to face the control module 20 when the delivery module 10 is coupled to the control module 20. The first interface surface 115 faces away with respect to the application surface 102.

The first interface surface 115 comprises a first interface portion 116. The first interface portion 116 is defined in the portion of the delivery module 10 that is more distal from the application surface 102 along the second direction D2.

The first interface portion 116 extends from one of the first gripping perimeter surfaces 110 to the other of the first gripping perimeter surfaces 110. The first interface portion 116 extends from the third perimeter surface 111 along the first direction D1. The first interface portion 116 partially delimits the housing compartment 101 on the opposite side with respect to the base wall 103 of the application surface 102.

The first interface portion 116 has a concave profile defined by two opposite surfaces 117 parallel to the two first gripping perimeter surfaces 110 and a connection surface 118 parallel to the third perimeter surface 111.

Each of the two opposite surfaces 117 of the concave profile of the first interface portion 116 is closer to the respective first gripping perimeter surface 110 than to the first central plane of the delivery module 10.

The connection surface 118 of the concave profile of the first interface portion 116 is closer to the third perimeter surface 111 than to a centreline plane of the delivery module 10 extended along the second direction D2 and the third direction D3.

As shown in FIG. 2, the first interface surface 115 further comprises a second interface portion 120 substantially parallel to the application surface 102. The second interface portion 120 is positioned at a smaller distance from the application surface 102 than the first interface portion 116.

The second interface portion 120 extends along the third direction D3 from one of the short sides to the opposite short side of the rectangular profile of the base wall 103 and, along the first direction D1, from the opposite long side to the third perimeter surface 111 towards the third perimeter wall 111.

The second interface portion 120 does not extend between the first gripping perimeter surfaces 110.

Still with reference to FIG. 2, the first interface surface 115 further comprises a third interface portion 121 extending from the second interface portion 120 to the first interface portion 116 and from one of the first gripping perimeter surfaces 110 to the other of the first gripping perimeter surfaces 110. The third interface portion 121 delimits the housing compartment 101 on the opposite side with respect to the third perimeter surface 111.

The third interface portion 121 comprises respective ends 122 placed at the first gripping perimeter surfaces 110 and a projecting portion 123 placed between the ends 122 and projecting on the opposite side with respect to the third perimeter surface 111.

The first interface surface 115 further comprises a first circular portion 124. The first circular portion 124 is configured to allow a driving rotor provided in the control module 20 to rotatably actuate by magnetic coupling a magnetic rotor provided in the delivery module 10 at the first circular portion 124 when the control module 20 is coupled to the delivery module 10.

The first circular portion 124 is placed between the first interface portion 116 and the third interface portion 121. The first circular portion 124 partially delimits the housing compartment 101 on the opposite side with respect to the application surface 102 and defines a recess with respect to the first interface portion 116.

The first interface surface 115 further comprises an inspection window 125 (FIGS. 2 and 4) configured to allow a visual inspection of the cartridge when the control module 20 is decoupled from the delivery module 10.

The inspection window 125 is placed at the first interface portion 116, overlapping the housing compartment 101. The inspection window 125 is parallel to the application surface 102.

The inspection window 125 is placed on the opposite side of the housing compartment 101 with respect to the light-permeable portion 107. In particular, the inspection window 125 and the light-permeable portion 107 are mutually overlapped. More particularly, the inspection window 125 and the light-permeable portion 107 are arranged such that the cartridge is positioned therebetween.

The light-permeable portion 107 is greater than the inspection window 125.

The inspection window 125 is configured to be covered by the control module 20, when the control module 20 is coupled to the delivery module 10, so as to prevent the visual inspection of the cartridge.

The control module 20 is, in some instances, in a colour and/or material different from that of the delivery module 10, so as to help the user to distinguish the two modules during the coupling.

The control module 20 in turn comprises a housing inside which a control unit is housed, which is usually made of a printed circuit and configured to control the delivery of the medicament from the delivery module 10, and a power supply battery configured to power the control unit.

As shown in FIGS. 1 and 5, the control module 20 comprises a top surface 202 extending substantially parallel to the application surface 102 when the control module 20 is coupled to the delivery module 10.

The top surface 202 has a substantially rectangular shape, with rounded corners, specular to the shape of the base wall 103. Therefore, similarly to the latter, the top surface 202 has two short sides oriented in the first direction D1 and two long sides oriented in the third direction D3.

The control module 20 comprises two opposite second gripping perimeter surfaces 210. The opposite second gripping perimeter surfaces 210 extend along the first direction D1 and along the second direction D2. The second gripping perimeter surfaces 210 are adjacent to the top surface 202 and partially delimit the aforementioned housing on opposite sides. The second gripping perimeter surfaces 210 are placed at opposite short sides of the top surface 202 and form with the top surface 202 an angle substantially equal to 90°. The second gripping perimeter surfaces 210 extend along the first direction D1 on a same long side of the top surface 202 towards the opposite long side moving on the respective short side. The second gripping perimeter surfaces 210 extend along the first direction D1 for about half the extent of the short sides of the top surface 202. The second gripping perimeter surfaces 210 extend from the top surface 202 along the second direction D2 away with respect to the top surface 202, i.e. towards the application surface 102 when the control module 20 is coupled to the delivery module 10.

The second gripping perimeter surfaces 210 have dimensions substantially equal to one another along the first direction D1 and the second direction D2. In particular, the two second gripping perimeter surfaces 210 are specular with respect to a second central plane of the control module 20 extending along the first direction D1 and the second direction D2 and coinciding with the first central plane of the delivery module 10 when the control module 20 is coupled to the delivery module 10.

Furthermore, the two first gripping perimeter surfaces 110 and the two second gripping perimeter surfaces 210 have substantially equal dimensions along the first direction D1.

When the control module 20 is coupled to the delivery module 10, each first gripping perimeter surface 110 is adjacent to and aligned with, or flush with, a respective second gripping perimeter surface 210 to define a substantially continuous side surface of the device 1.

As shown in FIGS. 1, 5-7, the control module 20 comprises a fourth perimeter surface 211 extending along the second direction D2 and the third direction D3. The fourth perimeter surface 211 is adjacent to the top surface 202 and partially delimits the aforementioned housing. The fourth perimeter surface 211 is placed at one of the long sides of the top surface 202 and forms with the top surface 202 an angle substantially equal to 90°. The fourth perimeter surface 211 extends from the top surface 202 along the second direction D2 away from the top surface 202, i.e. towards the application surface 102 when the control module 20 is coupled to the delivery module 10. The fourth perimeter surface 211 extends along the third direction D3 from a second gripping perimeter surface 210 to the other second gripping perimeter surface 210 moving on the respective long side of the top surface 202. The fourth perimeter surface 211 has a dimension substantially equal to the dimension of the second gripping perimeter surfaces 210 in the second direction D2.

Each second gripping perimeter surface 210 is joined to the fourth perimeter surface 211 by a rounded joining portion 212.

When the control module 20 is coupled to the delivery module 10, the fourth perimeter surface 211 is arranged substantially parallel to the third perimeter surface 111, on the opposite side to the latter.

The sum of the dimensions of any one of the first gripping perimeter surfaces 110 and of any one of the second gripping perimeter surfaces 210 along the first direction D1 is less than the dimension of the third perimeter surface 111 and of the fourth perimeter surface 211 along the third direction D3.

As shown in FIGS. 6 and 7, the control module 20 comprises a second interface surface 215 configured to be arranged so as to face the first interface surface 115 of the delivery module 10 when the delivery module 10 is coupled to the control module 20. The second interface surface 215 is substantially counter-shaped to the first interface surface 115 and faces away with respect to the top surface 202.

The second interface surface 215 comprises a first interface portion 216 substantially parallel to the top surface 202. The first interface portion 216 of the control module 20 is substantially counter-shaped to the second interface portion 120 of the delivery module 10 and is configured to be arranged so as to face the latter. The first interface portion 216 is defined in the portion of the control module 20 that is more distal from the top surface 202 along the second direction D2. The first interface portion 216 extends from one of the second gripping perimeter surfaces 210 to the other second gripping perimeter surface 210. The first interface portion 216 is adjacent to the fourth perimeter surface 211 and extends from the fourth perimeter surface 211 along the first direction D1. The first interface portion 216 partially delimits the aforementioned housing on the opposite side with respect to the top surface 202.

The second interface surface 215 further comprises a second interface portion 220 placed at a smaller distance from the top surface 202 than the first interface portion 216. The second interface portion 220 is substantially counter-shaped to the first interface portion 116 of the delivery module 10 and is configured to be arranged so as to face the latter. The second interface portion 220 extends along the third direction D3 from one of the short sides to the opposite short side of the rectangular profile of the top surface 202 and, along the first direction D1, from the long side opposite to the fourth perimeter surface 211 towards the fourth perimeter wall 211.

The second interface portion 220 has a convex profile, substantially specular to the concave profile of the first interface portion 116. The convex profile of the second interface portion 220 is defined by two opposite surfaces 217 parallel to the two first gripping perimeter surfaces 110 and a connection surface 218 parallel to the fourth perimeter surface 211. Each of the two opposite surfaces 217 of the convex profile of the second interface portion 220 is closer to the respective second gripping perimeter surface 210 than to the second central plane of the control module 20.

The connection surface 118 of the concave profile of the first interface portion 116 and the connection surface 218 of the convex profile of the second interface portion 220 are closer to the third perimeter surface 111 than to a center plane of the device 1 extending along the second direction D2 and the third direction D3 when the control module 20 is coupled to the delivery module 10.

The second interface surface 215 further comprises a third interface portion 221 extending from the second interface portion 220 to the first interface portion 216 and from one of the second gripping perimeter surfaces 210 to the other of the second gripping perimeter surfaces 210. The third interface portion 221 delimits the aforementioned housing on the opposite side with respect to the fourth perimeter surface 211. The third interface portion 221 of the control module 20 is substantially counter-shaped to the third interface portion 121 of the delivery module 10 and is configured to be arranged so as to face the latter. The third interface portion 221 comprises respective ends 222 placed at the second gripping perimeter surfaces 210 and a recessed portion 223 placed between the ends 222 and which is recessed towards the fourth perimeter surface 211.

The second interface surface 215 further comprises a second circular portion 224. The second circular portion 224 of the control module 20 is substantially counter-shaped to the first circular portion 124 of the delivery module 10 and is configured to be arranged so as to face the latter. The first circular portion 124 and the second circular portion 224 are configured to allow a precise alignment between the driving rotor provided in the control module at the second circular portion 224 and the magnetic rotor provided in the delivery module 10 at the first circular portion 124 when the control module 20 is coupled to the delivery module 10. The second circular portion 224 is placed between the second interface portion 220 and the third interface portion 221 and defines a protrusion with respect to the second interface portion 220.

The second interface surface 215, in particular the second interface portion 220 of the second interface surface 215, is configured to cover the inspection window 125 when the control module 20 is coupled to the delivery module 10 so as to prevent a visual inspection of the cartridge.

One or more gaskets are interposed between at least part of the first interface surface 115 and at least part of the second interface surface 215 in order to ensure the tightness of the coupling between the delivery module 10 and the control module 20.

As shown in FIG. 2, the delivery module 10 comprises opposite coupling recesses 130 extending along the first direction D1. The coupling recesses 130 are placed close to the first gripping perimeter surfaces 110. In particular, the coupling recesses 130 are placed at the first interface portion 116. The coupling recesses 130 extend starting from the opposite surfaces 117 of the concave profile of the first interface portion 116 towards the first gripping perimeter surfaces 110.

As shown in FIG. 5, the control module 20 comprises opposite coupling projections 230 extending along the first direction D1. The coupling projections 230 are configured to slide in the coupling recesses 130 during the coupling of the control module 20 to the delivery module 10 so as to guide the sliding thereof along the coupling direction A1. The coupling projections 230 are placed close to the second gripping perimeter surfaces 210.

As shown in FIGS. 6 and 7, the control module 20 comprises a locking projection 300. The locking projection 300 extends from the second interface surface 215 of the control module 20 along a development direction perpendicular to the coupling direction A1 and parallel to the second direction D2. More particularly, the locking projection 300 extends from the first interface portion 216 of the second interface surface 215 along the aforementioned development direction. When the control module 20 is coupled to the delivery module 10, the locking projection 300 projects towards the application surface 102.

The locking projection 300 comprises a stem 301 extending along the aforementioned development direction from the second interface surface 215 and having an end 302, and a tooth 303 extending from the end 302 parallel to the coupling direction A1.

The locking projection 300 comprises a push portion 304 extending along the aforementioned development direction and a hooking portion 305 extended from the push portion 304 along a direction parallel to the coupling direction A1, i.e. parallel to the first direction D1. The hooking portion 305 comprises a surface placed on the tooth 303, substantially parallel to the application surface 102, and facing towards the second interface surface 215.

The locking projection 300 is placed at the second central plane of the control module 20. The control module 20 further comprises a closing projection 310 extending along a development direction parallel to the development direction of the locking projection 300. In particular, the closing projection 310 extends along the second direction from the second interface surface 215. More particularly, the closing projection 310 extends from an edge of mutual joining of the second interface surface 215 and of the fourth perimeter surface 211, flush with the fourth perimeter surface 211. The closing projection 310 is placed at the second central plane of the control module 20 and is spaced from the locking projection 300 along the coupling direction A1.

As shown in FIGS. 2, 8-10, the delivery module 10 comprises a seat 400 configured to cooperate with the locking projection 300 during the coupling between the delivery module 10 and the control module 20.

The seat 400 comprises an inlet 401 configured to receive the locking projection 300. The inlet 401 is placed on the first interface surface 115, in particular on the second interface portion 120 of the first interface surface 115, at one of the long sides of the base wall 103.

The seat 400 further comprises an internal volume 402 configured to house the locking projection 300 at the end of the coupling between the delivery module 10 and the control module 20. The internal volume 402 is contiguous with the inlet 401 along the coupling direction A1.

The seat 400 also comprises a coupling portion 403 overlapping the inlet 401 and the internal volume 402 (FIG. 2). The coupling portion 403 is defined between two opposite guide surfaces 404 extending from the inlet 401 to the internal volume 402 along the coupling direction A1. In particular, the guide surfaces 404 extend along the first direction D1 and the second direction D2 and are parallel to each other. The guide surfaces 404 are adjacent to the second interface portion 120 of the first interface surface 115. The guide surfaces 404 are configured to guide the locking projection 300 between the inlet 401 and the internal volume 402 during the coupling of the control module 20 with the delivery module 10.

The coupling portion 403 further comprises a stop surface 405 extending perpendicularly to the coupling direction A1 and facing the internal volume 402. The stop surface 405 extends between the two guide surfaces 404 along the third direction D3 and the second direction D2. The stop surface 405 is adjacent to the second interface portion 120 of the first interface surface 115 and defines an end stop for the locking projection 300 along the coupling direction A1.

The seat 400 further comprises a hooking surface configured to be arranged in abutment against the hooking portion 305 of the locking projection 300 when the locking projection 300 is housed in the internal volume 402, like in the configuration of FIG. 10. The hooking surface is substantially parallel to the application surface 102 and is faced towards the application surface 102.

As shown in FIGS. 8-10, the delivery module 10 further comprises a slider 410 associated with the seat 400 and slidably movable between a first position and a second position along a manoeuvring direction M1 perpendicular to the coupling direction A1 and parallel to the application surface 102. In particular, the manoeuvring direction M1 is parallel to the third direction D3.

The slider 410 comprises a main body 411 slidably mounted in the seat 400.

The main body 411 comprises a service portion 412 shaped to obstruct the inlet 401 when the slider 410 is in the first position and not to obstruct the inlet 401 when the slider 410 is in the second position. When the slider 410 is in the first position, the service portion 412 is arranged at the coupling portion 403. When the slider 410 is in the second position, the service portion 412 is arranged in a position that is adjacent to the coupling portion 403.

The service portion 412 comprises a push surface 413 facing the inlet 401 when the slider 410 is in the first position. The push surface 413 is oriented obliquely with respect to the coupling direction A1. In particular, the push surface 413 is oriented according to a plane perpendicular to the application surface 102 and oriented obliquely at an angle with respect to the third direction D3 which is, in some instances, comprised between 30° and 60°, even more preferably between 40° and 50°, for example equal to about 45°.

The service portion 412 further comprises a locking surface 414 facing the internal volume 402 when the slider 410 is in the first position. The locking surface 414 is oriented perpendicularly with respect to the coupling direction A1. The locking surface 414 is parallel to the stop surface 405. In particular, the locking surface 414 extends along the second direction D2 and the third direction D3.

The slider 410 further comprises a manoeuvring portion 416, shown in FIG. 3, which is manually operable along the manoeuvring direction M1 to shift the slider 410 from the first position to the second position. The manoeuvring portion 416 projects from the main body 411 in the second direction D2 towards the application surface 102 until it is arranged outside the seat 400 through an opening 417 formed in the application surface 102.

The manoeuvring portion 416 is arranged substantially flush with the application surface 102 and comprises a friction surface 418 parallel to the application surface 102 and configured to allow a user to move the slider 410 from the first position to the second position parallel to the friction surface 418 by his/her finger. In some instances, the friction surface 418 has a high roughness or surface asperities configured to increase the friction with the user's finger. The manoeuvring portion 416 is positioned at the application surface 102 such that, when the device 1 is applied on the patient's body by the application surface 102, the manoeuvring portion 416 is unreachable and cannot be reached.

An elastic element 419 is interposed between a first shoulder 420 formed in the seat 400 and a second shoulder 421 formed in the slider 410 so as to exert an elastic force against the slider 410. In some instances, the elastic element 419 is a spring.

When the slider 410 is in the first position, the elastic force of the elastic element 419 retains it in the first position, both before the coupling between the delivery module 10 and the control module 20 and at the end of the coupling between the delivery module 10 and the control module 20. In order to shift the slider from the first position to the second position, the user exerts on the manoeuvring portion 416 a force along the manoeuvring direction M1 such as to overcome the elastic force of the elastic element 419. When the slider is in the second position, the elastic element 419 pushes it towards the first position.

In order to couple the control module 20 to the delivery module 10 the two modules are moved towards each other until the coupling projections 230 are inserted into the coupling recesses 130 and the locking projection 300 into the seat 400 through the inlet 401. At this point, the control module 20 is made to slide towards the delivery module 10 along the coupling direction A1.

As shown in FIGS. 8-10, the push portion 304 of the locking projection 300 engages in abutment the push surface 413 of the slider 410, which is in the first position. Thanks to the oblique orientation of the push surface 413, the push portion 304, while moving along the coupling direction A1, shifts the slider 410 from the first position to the second position and slides on the push surface 413, overcoming the elastic push exerted on the slider 410 by the elastic element 419. In this way, the inlet 401 can be accessed and the locking projection 300 can enter into the internal volume 402. Alternatively, the slider 410 may be moved from the first position to the second position by manually operating the manoeuvring portion 416.

By continuing to move along the coupling direction A1, the push portion 304 disengages the push surface 413 and the locking projection 300 reaches the internal volume 402. The slider 410 is thus free to return to the first position by effect of the return action exerted by the elastic element 419, so that the service portion 412 of the slider 410 obstructs the inlet 401.

The locking projection 300, when housed in the internal volume 402, is surrounded by: the stop surface 405, the guide surfaces 404 and the locking surfaces 414 and can no longer move in the coupling direction A1 in either direction. Furthermore, when the locking projection 300 is housed in the internal volume 402 the hooking portion 305 overlaps the hooking surface and prevents relative movements of the delivery module 10 and of the control module 20 perpendicularly to the application surface 102. The control module 20 is thus stably coupled to the delivery module 10.

In this configuration, the closing projection 310 is positioned so as to close the inlet 401, as shown in FIG. 10.

What is claimed is:

1. A device for subcutaneous delivery of a medicament, comprising:
a delivery module configured to house a cartridge containing a medicament to be delivered to a patient via a needle; and
a control module reversibly couplable to the delivery module and configured to house at least one control unit and a battery to power the control unit;
wherein the delivery module comprises:
an application surface configured to contact the patient; and
two opposite first gripping perimeter surfaces extending along a first direction parallel to the application surface and along a second direction perpendicular to the application surface, wherein the two first gripping perimeter surfaces have substantially equal dimensions in the second direction;
wherein the control module comprises:
two opposite second gripping perimeter surfaces extending along the first direction and along the second direction, wherein the two second gripping perimeter surfaces have substantially equal dimensions in the second direction, and
wherein the two first gripping perimeter surfaces and the two second gripping perimeter surfaces have substantially equal dimensions in the first direction.

2. The device of claim 1, wherein the delivery module comprises opposite coupling recesses extending along the first direction proximate the first gripping perimeter; surfaces and
the control module comprises opposite coupling projections extending along the first direction proximate the second gripping perimeter surfaces and configured to slide in the coupling recesses during the coupling of the control module to the delivery module.

3. The device of claim 1, wherein:
the two first gripping perimeter surfaces are specular with respect to a first central plane of the delivery module extending along the first direction and the second direction;
the two second gripping perimeter surfaces are specular with respect to a second central plane of the control module extending along the first direction and the second direction,
wherein when the control module is coupled to the delivery module the first central plane coincides with the second central plane.

4. The device of claim 3, wherein:
the delivery module comprises a third perimeter surface extending between the two first gripping perimeter surfaces along the second direction and along a third direction that is parallel to the application surface and perpendicular to the first direction;
the control module comprises a fourth perimeter surface extending between the two second gripping perimeter surfaces along the second direction and the third direction, the fourth perimeter surface being parallel to the third perimeter surface when the control module is coupled to the delivery module; and
the sum of the dimensions of any one of the first gripping perimeter surfaces and of any one of the second gripping perimeter surfaces along the first direction is smaller than the dimension of the third perimeter surface and of the fourth perimeter surface along the third direction.

5. The device of claim 4, wherein:
the delivery module comprises a first interface surface;
the control module comprises a second interface surface substantially counter-shaped to the first interface surface, the second interface surface configured to be arranged so as to face the first interface surface when the control module is coupled to the delivery module;
the first interface surface comprises a first interface portion extending from one of the first gripping perimeter surfaces to the other first gripping perimeter surface, the first interface having a concave profile; and
the second interface surface comprises a second interface portion having a convex profile, the second interface portion configured to be arranged so as to face the first interface portion when the control module is coupled to the delivery module.

6. The device of claim 5, wherein:
the concave profile is defined by two opposite surfaces parallel to the two first gripping perimeter surfaces and a connection surface parallel to the third perimeter surface;
the convex profile is defined by two opposite surfaces parallel to the two second gripping perimeter surfaces and a connection surface parallel to the fourth perimeter surface.

7. The device of claim 6, wherein:
each of the two opposite surfaces of the concave profile is closer to a respective first gripping perimeter surface than to the first central plane of the delivery module;
each of the two opposite surfaces of the convex profile is closer to the first gripping perimeter surface than to the second central plane of the control module when the control module is coupled to the delivery module.

8. The device of claim 6, wherein the connection surface of the concave profile and the connection surface of the convex profile are closer to the third perimeter surface than a center plane of the device extending along the second direction and the third direction when the control module is coupled to the delivery module.

9. The device of claim 5, wherein the first interface portion is defined in a top portion of the delivery module and the second interface portion is defined in a top portion of the control module.

10. The device of claim 5, wherein the first interface surface comprises a first circular portion and the second interface surface comprises a second circular portion configured to overlap the first circular portion when the control module is coupled to the delivery module.

11. The device of claim 10, wherein the first circular portion defines a recess in the delivery module and the second circular portion defines a protrusion in the control module.

12. The device of claim 5, wherein the first interface surface comprises a second interface portion substantially parallel to the application surface and a third interface portion extending from the second interface portion away from the application surface up to the first interface portion.

13. The device of claim 12, wherein the first interface portion, the second interface portion and the third interface portion of the first interface surface have substantially equal dimensions in the third direction.

14. The device of claim 12, comprising at least one gasket interposed between at least part of the first interface surface and at least part of the second interface surface.

15. The device of claim 1, wherein the control module has a colour different from that of the delivery module.

16. The device of claim 1, wherein the control module has a surface finish different from that of the delivery module.

17. The device of claim 2, wherein:
the two first gripping perimeter surfaces are specular with respect to a first central plane of the delivery module extending along the first direction and the second direction;
the two second gripping perimeter surfaces are specular with respect to a second central plane of the control module extending along the first direction and the second direction;
wherein when the control module is coupled to the delivery module the first central plane coincides with the second central plane.

18. The device of claim 1, wherein:
the delivery module comprises a third perimeter surface extending between the two first gripping perimeter surfaces along the second direction and along a third direction that is parallel to the application surface and perpendicular to the first direction;
the control module comprises a fourth perimeter surface extending between the two second gripping perimeter surfaces along the second direction and the third direction, the fourth perimeter surface being parallel to the third perimeter surface when the control module is coupled to the delivery module; and
the sum of the dimensions of any one of the first gripping perimeter surfaces and of any one of the second gripping perimeter surfaces along the first direction is smaller than the dimension of the third perimeter surface and of the fourth perimeter surface along the third direction.

19. The device of claim 1, wherein:
the delivery module comprises a first interface surface;
the control module comprises a second interface surface substantially counter-shaped to the first interface surface and configured to be arranged so as to face the first interface surface when the control module is coupled to the delivery module;
the first interface surface comprises a first interface portion extending from one of the first gripping perimeter surfaces to the other first gripping perimeter surface and having a concave profile; and
the second interface surface comprises a second interface portion having a convex profile and configured to be arranged so as to face the first interface portion when the control module is coupled to the delivery module.

20. The device of claim 7, wherein the connection surface of the concave profile and the connection surface of the convex profile are closer to the third perimeter surface than a center plane of the device extending along the second direction and the third direction when the control module is coupled to the delivery module.
